(19) United States
(12) Patent Application Publication
(10) Pub. No.: US 2016/0128337 A1
(43) Pub. Date: May 12, 2016

(54) ANTIBACTERIAL COMPOSITIONS AND METHODS OF USE

(71) Applicant: BiOWiSH Technologies, Inc., Cincinnati, OH (US)

(72) Inventors: Richard S. Carpenter, West Chester, OH (US); JoElla Barnes, Arcola, IL (US); John Gorsuch, Cincinnati, OH (US)

(21) Appl. No.: 14/936,288

(22) Filed: Nov. 9, 2015

Related U.S. Application Data
(60) Provisional application No. 62/076,663, filed on Nov. 7, 2014.

(51) Int. Cl. A01N 63/00; A61K 35/744; A61K 35/747; A01N 25/34

(52) U.S. Cl. CPC A01N 63/00; A01N 25/34; A61K 35/744; A61K 35/747

(57) ABSTRACT

The present invention relates to antimicrobial compositions useful in bodies of water and in particular raceway systems used to raise aquatic organisms.

### RELATED APPLICATIONS

This application claims priority to and benefit of U.S. Provisional Application No. 62/076,663, filed in Nov. 7, 2014, the contents of which are hereby incorporated by reference in their entireties.

### FIELD OF THE INVENTION

The present invention relates to water treatment compositions containing micro-organisms and methods of using the compositions.

### BACKGROUND OF THE INVENTION

The use of antimicrobial agents to kill or prevent the growth of undesirable organisms has been studied extensively. In particular, antimicrobial agents such as fungicides, antiviral, and antibacterial compounds have been examined. Although a number of antimicrobial agents are effective, they have drawbacks. For example, they can be very toxic and difficult to handle and not environmentally friendly, which limits their use. Thus, it would be desirable to have an antimicrobial agent that can be used in aquatic applications. Described herein are methods and compositions that address the shortcomings of current antimicrobial agents.

### SUMMARY OF THE INVENTION

In various aspects the invention provides compositions containing a mixture of micro-organisms for the treatment of a body of water.

In one aspect the invention provides an antibacterial composition containing a mixture of bacteria selected from the family Lactobacillaceae. Preferably, the mixture of bacteria contains a species selected from the genus Pediococcus and Lactobacillus. Most preferably the composition contains a mixture of bacteria comprises equal amounts of Pediococcus acidilactici, Pediococcus pentosaceus, and Lactobacillus plantarum. Each of the bacteria in the mixture is individually anaerobically fermented, harvested, dried, and ground to produce a powder having a mean particle size of about 200 microns, with greater than 60% of the mixture in the size range between 100-800 microns.

In some embodiments, the antibacterial composition further comprises a species selected from the genus Bacillus. In some embodiments, the Bacillus species is Bacillus subtilis 34KLB.

In some embodiments, the antibacterial composition further contains a species selected from the genus Bifidobacterium. For example the Bifidobacterium is Bifidobacterium animalis.

In some embodiments, the composition contains equal amounts of Pediococcus acidilactici, Pediococcus pentosaceus, Lactobacillus plantarum, Bifidobacterium animalis, Bacillus subtilis, Bacillus amyloliquefaciens, Bacillus licheniformis, Bacillus pumilus, Bacillus coagulans, Bacillus megaterium, and Paenibacillus polymyxa, wherein each bacteria in the mixture is individually fermented under conditions optimum for growth, harvested, dried, and ground to produce a powder having a mean particle size of about 200 microns, with greater than 60% of the mixture in the size range between 100-800 microns.

In some embodiments, the composition comprises about 1%, 2%, 3%, 4%, 5%, 6%. 7%. 8%, 9%, 10%, 15%, 20%, 25% or more by weight of a mixture comprising Pediococcus acidilactici, Pediococcus pentosaceus, and Lactobacillus plantarum, where each of the bacteria are present in the mixture in equal amounts by weight. Optionally, the composition further includes about 0.1%, 0.15%, 0.2%, 0.3%, 0.4%, 0.5%, 0.6%, 0.7%, 0.8%, 0.9%, 1%, 2%, 3%, 4%, 5% or 10% or more of Bacillus subtilis 34KLB by weight. Each bacterium in the mixture is individually fermented under conditions optimum for growth, harvested, dried, and ground to produce a powder having a mean particle size of about 200 microns, with greater than 60% of the mixture in the size range between 100-800 microns.

In a preferred embodiment the composition comprises about 2% by weight of by weight of a mixture comprising Pediococcus acidilactici, Pediococcus pentosaceus, and Lactobacillus plantarum, where each of the bacteria are present in the mixture in equal amounts by weight and 0.15% f Bacillus subtilis 34KLB by weight. Each bacterium in the mixture is individually fermented under conditions optimum for growth, harvested, dried, and ground to produce a powder having a mean particle size of about 200 microns, with greater than 60% of the mixture in the size range between 100-800 microns.

In a preferred embodiment the composition comprises about 10% by weight of by weight of a mixture comprising Pediococcus acidilactici, Pediococcus pentosaceus, and Lactobacillus plantarum, where each of the bacteria are present in the mixture in equal amounts by weight and 0.15% f Bacillus subtilis 34KLB by weight. Each bacterium in the mixture is individually fermented under conditions optimum for growth, harvested, dried, and ground to produce a powder having a mean particle size of about 200 microns, with greater than 60% of the mixture in the size range between 100-800 microns.

In some embodiments, the composition has a moisture content of less than about 5%; and a final bacterial concentration of about between 10⁵-10¹¹ colony forming units (CFU) per gram of the composition.

In various aspects the composition further contains an inert carrier such as anhydrous dextrose, dextrose monohydrate, dendritic salt, rice bran, wheat bran, oat bran, soybean meal, rice hulls, Nutri-Sure or a mixture thereof. Preferably, the inert carrier is at a concentration of about between 75-99% (w/w), e.g., 75-95% (w/w).

Also included in the invention are methods of treating a body of water by contacting said body of water with the composition according to the invention. The composition is dosed between 0.001 and 100 mg/L. In preferred aspects the body of water is a lagoon, pond, lake, or raceway system used to raise aquatic organisms. The aquatic organism is shrimp or fin fish. Fin fish include for example catfish, tilapia, salmon, carp, sea bass, or cod.

Treatment of the body of water results in a decrease in pathogenic bacteria. The decrease is at least a 2-log reduction in the population of pathogenic bacteria. The pathogenic bacteria are Vibrio, Escherichia, Listeria, or Salmonella. For example, the pathogenic bacteria are V. cholera, V. parahaemolyticus, V. harveyi, V. vulnificans, or V. fischeri.

Also included in the invention are methods of treating an aquaculture system infected with V. parahaemolyticus, wherein the system is dosed with between 0.001 and 100 mg/L of the composition according to the invention. Treatment of the body of water results in a decrease of V. parahaemolyticus.

Further included in the invention is a method for managing Early Mortality Syndrome in shrimp farming, by exposing the shrimp to the composition of the invention at concentrations of between 0.001 and 100 mg/L. The shrimp are exposed to the composition by dosing directly to the water or through a feed particle. The feed particle is produced through a standard pelleting process. Alternatively the feed particle is prepared by coating the bacterial composition of the invention onto a feed particle. In other embodiments the bacterial composition of the invention is admixed into the feed.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below.

All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a graph showing the inhibition of V. parahaemolyticus by the compositions of the invention.

FIG. 2 is a graph showing inhibition of Vibrio fisheri by the microbial composition in Example 2B.

### DETAILED DESCRIPTION OF THE INVENTION

The invention provides anti-bacterial microbial compositions for treating and preventing bacterial contamination in bodies of water such as lagoons, ponds, lakes or raceway systems used to raise aquatic organisms. The anti-bacterial compositions are useful in preventing and treating diseases caused by pathogenic bacteria in the aquatic organisms.

In some aspects the microbial compositions contain a mixture of bacteria selected from the family Lactobacillaceae. For example, the bacteria are selected from the genus Pediococcus and Lactobacillus. Preferably, the mixture contains Pediococcus acidilactici, Pediococcus pentosaceus, and Lactobacillus plantarum. In some embodiments, the microbial composition comprises a species selected from the genus Bacillus. For example, the Bacillus species is Bacillus subtilis 34KLB. In some embodiments, the microbial compositions include a bacterial species selected from the genus Bifidobacterium. For example the Bifidobacterium is Bifidobacterium animalis.

Importantly, the composition fully disperses upon the addition to water and unlike other water treatment microbial compositions, the composition described herein does not require a pre-activation of the bacteria prior to use.

The microbial compositions decrease the concentration of pathogenic bacteria in the water. Preferably, there is at least a 2-log reduction in the population of pathogenic bacteria.

The terms “microbial”, “bacteria” or “microbes” as used herein, refers to microorganisms that confer a benefit. The microbes according to the invention may be viable or non-viable. The non-viable microbes are metabolically-active. By “metabolically-active” is meant that they exhibit at least some residual enzyme, or secondary metabolite activity characteristic to that type of microbe.

By the term “non-viable” as used herein is meant a population of bacteria that is not capable of replicating under any known conditions. However, it is to be understood that due to normal biological variations in a population, a small percentage of the population (i.e. 5% or less) may still be viable and thus capable of replication under suitable growing conditions in a population which is otherwise defined as non-viable.

By the term “viable bacteria” as used herein is meant a population of bacteria that is capable of replicating under suitable conditions. A (population of bacteria that does not fulfill the definition of “non-viable” (as given above) is considered to be “viable”.

By the term “aquatic organisms” as used herein is meant to include shrimp or fin fish. Fin fish include for example, catfish, tilapia, salmon, carp, sea bass, or cod.

“Treating” as used herein is meant inoculating water with microbes designed to enhance efficient degradation of organic matter.

Unless stated otherwise, all percentages mentioned document are by weight based on the total weight of the composition.

The microbes used in the product according to the present invention may be any conventional mesophilic bacteria. It is preferred that the bacteria are selected from the Lactobacillacae families. More preferably the bacteria are selected form the genus Bacillus and Lactobacillus. Optionally, the composition further contains a bacteria selected from Bifidobacterium. For example, the Bifidobacterium is Bifidobacterium animalis.

In a preferred composition, the mixture contains Pediococcus acidilactici, Pediococcus pentosaceus, and Lactobacillus plantarum. In another preferred composition the mixture contains Pediococcus acidilactici, Pediococcus pentosaceus, Lactobacillus plantarum, Bifidobacterium animalis, Bacillus subtilis, Bacillus amyloliquefaciens, Bacillus licheniformis, Bacillus pumilus, Bacillus coagulans, Bacillus megaterium, and Paenibacillus polymyxa.

In some embodiments, the composition comprises about 1%, 2%, 3%, 4%, 5%, 6%. 7%. 8%, 9%, 10%, 15%, 20%, 25% or more by weight of a mixture comprising Pediococcus acidilactici, Pediococcus pentosaceus, and Lactobacillus plantarum, where each of the bacterium is present in the mixture in equal amounts by weight. Optionally, the composition further includes about 0.1%, 0.15%, 0.2%, 0.3%, 0.4%, 0.5%, 0.6%, 0.7%, 0.8%, 0.9%, 1%, 2%, 3%, 4%, 5% or 10% or more of Bacillus subtilis 34KLB by weight. Each bacterium in the mixture is individually fermented under conditions optimum for growth, harvested, dried, and ground to produce a powder having a mean particle size of about 200 microns, with greater than 60% of the mixture in the size range between 100-800 microns.

In a preferred embodiment, the composition comprises about 2% by weight of a mixture comprising Pediococcus acidilactici, Pediococcus pentosaceus, and Lactobacillus plantarum, where each of the bacterium is present in the mixture in equal amounts by weight and 0.15% f Bacillus subtilis 34KLB by weight. In some embodiments, this preferred composition further includes 4% diatomaceous earth. Each bacterium in the mixture is individually fermented under conditions optimum for growth, harvested, dried, and ground to produce a powder having a mean particle size of about 200 microns, with greater than 60% of the mixture in the size range between 100-800 microns.

In a preferred embodiment, the composition comprises about 10% by weight of a mixture comprising Pediococcus acidilactici, Pediococcus pentosaceus, and Lactobacillus plantarum, where each of the bacterium is present in the mixture in equal amounts by weight and 0.15% f Bacillus subtilis 34KLB by weight. Each bacterium in the mixture is individually fermented under conditions optimum for growth, harvested, dried, and ground to produce a powder having a mean particle size of about 200 microns, with greater than 60% of the mixture in the size range between 100-800 microns.

The levels of the bacteria to be used according to the present invention will depend upon the types thereof. Preferably, that each of the individual species of bacteria in the mixture is present in equal amounts. It is preferred that the present product contains bacteria in an amount between about 10⁵ and 10¹¹ colony forming units per gram.

The bacteria according to the invention may be produced using any standard fermentation process known in the art. For example, solid substrate or submerged liquid fermentation. The fermented cultures can be mixed cultures or single isolates.

In some embodiments the bacteria are anaerobically fermented in the presence of carbohydrates. Suitable carbohydrates include inulin, fructo-oligosaccharide, and gluco-oligosaccharides.

The bacterial compositions are in powdered, dried form. Alternatively, the bacterial compositions are in liquid form.

After fermentation the bacteria are harvested by any known methods in the art. For example the bacteria are harvested by filtration or centrifugation.

The bacteria are dried by any method known in the art. For example the bacteria are air dried, or dried by freezing in liquid nitrogen followed by lyophilization.

The compositions according to the invention have been dried to moisture content less than 20%, 15%, 10%, 9%, 8%, 7%, 6%, 5%, 4%, 3%, 2%, or 1%. Preferably, the composition according to the invention has been dried to moisture content less than 5%.

In some embodiments the dried powder is ground to decrease the particle size. The bacteria are ground by conical grinding at a temperature less than 10° C., 9° C., 8° C., 7° C., 6° C., 5° C., 4° C., 3° C., 2° C., 1° C., 0° C., or less. Preferably the temperature is less than 4° C.

The particle size is less than 1500, 1400, 1300, 1200, 1100, 1000, 900, 800, 700, 600, 500, 400, 300, 200, or 100 microns. Preferably, the freeze dried powder is ground to decrease the particle size such that the particle size is less than 800 microns. Most preferred are particle sizes less than about 400 microns. In most preferred embodiments, the dried powder has a mean particle size of 200 microns, with 60% of the mixture in the size range between 100-800 microns. In various embodiments the freeze dried powder is homogenized.

In various embodiments, the bacteria compositions are mixed with an inert carrier such as anhydrous dextrose, dextrose monohydrate, dendritic salt, rice bran, wheat bran, oat bran, soybean meal, rice hulls, Nutri-sure or a mixture thereof.

The inert carrier is at a concentration of at least 60%, 70%, 75%, 80%, 85%, 90%, 95% or more. Preferably, the inert carrier is at a concentration of about between 75-99% (w/w), such as 75-95% (w/w). More preferably, the inert carrier is present at least 90%, 91%, 95%, 96%, 97% or 98% (w/w).

In other aspects the bacterial compositions contain an organic emulsifier such as, for example, soy lecithin. The organic emulsifier is at a concentration of about 1%, 2%, 3%, 4%, 5%, 6%, 7%, 8%, 9% or 10%. Preferably, the organic emulsifier is at a concentration of between 2 to 5% (w/w).

Further, if desired, the bacterial compositions may be encapsulated to further increase the probability of survival; for example in a sugar matrix, fat matrix or polysaccharide matrix.

In some embodiments, the composition further comprises a drying agent such as diatomaceous earth. The drying agent can be at a concentration of about 1-10% (w/w), e.g., 1-9% (w/w), 1-8% (w/w), 1-7% (w/w), 1-6% (w/w), 1-5% (w/w). In some embodiments, the drying agent is at a concentration of about 4% (w/w).

The bacterial compositions of the invention are used to treat bodies of waters such as lagoons, pond, lakes, raceway systems used to raise aquatic organisms and the like.

Solutions of the compositions can be pumped into the material to be treated (liquid, sludge, or solid) or sprayed onto the surface, or into the airspace surrounding the material, or applied to a filter through which the water to be cleaned is passed. The dry material can be mixed into a slurry or solution at the point of application and applied in a similar manner.

The aqueous solution or the dry composition according to the invention can be employed to decrease pathogenic bacteria in the water. Preferably there is at least a 0.5 log, 1 log 2 log, 3 log, 4 log, 5 log or more reduction in the concentration of pathogenic bacteria. The pathogenic bacteria are, for example, Vibrio, Escherichia, Listeria, or Salmonella. In preferred embodiments the pathogenic bacteria is V. cholera, V. parahaemolyticus, V. harveyi, V. vulnificans, or V. fischeri.

In preferred methods, aquaculture systems infected with V. parahaemolyticus are treated by dosing the aquaculture systems with between 0.001 and 100 mg/L of the compositions of the invention.

In another preferred method early mortality syndrome (EMS) in shrimp is treated/managed by exposing the shrimp to any one of the compositions of the invention. Early Mortality Syndrome (EMS) is an emerging disease caused by bacteria. EMS typically affects shrimp that are not yet marketable size (40 days old or younger). The disease is often fatal to shrimp. Infected shrimp ponds can experience loss rates as high as 100 percent. Shrimp are exposed to the inventive composition by dosing the water directly or by providing the composition in the form of a feed.

The compositions of the invention are used to produce animal feed products and supplements or used as an animal feed additive. Although it is possible to achieve the benefits of the present invention by simply ad-mixing the anti-microbial compositions of the invention with animal feed or by using the compositions as a feed supplement, it is an object of the present invention to provide ready-to-eat feed products containing both a balanced diet ration and the anti-microbial compositions of the present invention.

Accordingly, the invention also provides feed product. The feed product can be provided as a dried powder or liquid.

The feed products can be produced by coating a pre-manufactured ready-to-eat animal feed product with the anti-microbial composition of the invention. Coating the animal feed product can be achieved by methods known in the art. For example, the dried compositions of the invention can be dispersed in a suitable oil or a low melting grease or wax to which an animal feed product is added, or alternatively the oil or molten grease or wax containing the anti-microbial compositions of the invention is sprayed onto the animal feed product.

Additionally, feed products containing the compositions of the invention may be prepared by mixing anti-microbial compositions of the invention with any suitable ingredients, such as those commonly used in the production of animal feed. The animal feed then may be produced in many different ways as desired. However, an especially suitable way to produce the feed products of the invention is by extrusion cooking. This can be done by methods well known in the art.

For example, in one suitable process, a feed mixture is fed into a pre-conditioner. The feed mixture is made up of a starch source and other ingredients such as sugar, salt, spices, seasonings, vitamins and minerals, flavoring agents, coloring agents, antioxidants, protein sources, yeast extracts, fats and the like.

Suitable starch sources are, for example, corn, rice, wheat, beets, barley, algae, soy and oats. The starch source may be a grain, a meal, gluten, or a flour.

Suitable protein sources may be selected from any suitable animal or vegetable protein source; for example meat meal, bone meal, fish meal, soy protein concentrates, milk proteins, gluten, yeast extracts, whey, and the like. The choice of the protein source will be largely determined by the nutritional needs, palatability considerations, and the type of feed product produced. Of course, the starch source may also be a source of protein.

If desired, sources of insoluble fiber may also be included; for example wheat bran, corn bran, rice bran, rye bran and the like. Further, if desired, a source of soluble fiber may be included, for example, chicory fibers, oat bran concentrate, guar gum, carob bean gum, xanthan gum, and the like.

Depending upon the desired form of the feed product, the starch content of the feed mixture may be varied. For example, for an expanded cereal product, the feed mixture preferably includes up to about 40% by weight of starch. However, for a flaked product, it is not necessary to use large amounts of starch in the feed mixture since it is possible to flake an unexpanded product.

In the pre-conditioner, water or steam, or both, is mixed into the feed mixture. Sufficient water or steam is mixed into the feed mixture to moisten the feed mixture. If desired, the temperature of the feed mixture may be raised in the pre-conditioner to about 60-90° C. It is not necessary to subject the feed mixture to preconditioning but it is advantageous to do so.

The moistened feed leaving the pre-conditioner is then fed into an extruder along with the antimicrobial composition. The extruder may be any suitable single or twin screw, cooking-extruder. Suitable extruders may be obtained from Wenger Manufacturing Inc., Clextral SA; Buhler AG, and the like. During passage through the extruder, the moistened feed passes through a cooking zone, in which it is subjected to mechanical shear and is heated; for example up to a maximum temperature of up to about 150° C. and a forming zone. The gauge pressure of the forming zone is about 300 KPa to about 10 MPa, as desired. If desired, water or steam, or both, may be introduced into the cooking zone. If desired, a small amount of edible oil may be fed into the extruder along with the moistened feed to facilitate the extrusion process or as a carrier for oil soluble additives. Any suitable oil may be used; for example vegetable oils such as sunflower oil, safflower oil, corn oil, and the like. If oils are used, oils which are high in mono-unsaturates are particularly preferred. Hydrogenated oils or fats are also preferred. The amount of oil used is preferably kept below about 1% by weight.

The food matrix leaving the extruder is forced through a suitable die. A shaped extrudate, which has a cross-sectional shape corresponding to that of the orifice of the die, leaves the die.

If a flaked product is to be produced, the pieces may then be transferred to a flaking apparatus. Suitable apparatus are well known and widely used in the cereal industry and may be purchased from, for example, Buhler AG in Switzerland. If desired, the pieces may be partially dried before flaking.

The pieces are then dried to a moisture content below about 10% by weight. This is conveniently carried out in a hot air drier as is conventional.

Numerous modifications may be made to the embodiments described above. For example, it is not necessary to produce the cereal product by extrusion cooking. Instead the cereal product may be produced by any suitable method of producing dried, ready-to-eat cereal pieces. For example, the feed materials may be cooked with water to provide a cooked paste. The paste is then roller-dried to produce dried flakes; usually of a thickness of about 0.6 to about 1 mm.

The compositions of the invention are manufactured by any method suitable for production of bacterial compositions. Preferably, mixtures of bacteria containing Lactobacillaceae, are manufactured by individually fermenting each organism; individually harvesting each organism; drying the harvested organism; grinding the dried organisms to produce a powder combining each of the organisms to produce a bacterial mixture.

A better understanding of the present invention may be given with the following examples which are set forth to illustrate, but are not to be construed to limit the present invention.

### EXAMPLES

### Example 1

### Preparation of the Microbial Species

The microbial mixture of the present invention may be made by any of the standard fermentation processes known in the art. In the following examples, both solid state and submerged liquid fermentation processes are described:

Solid State Fermentation

Individual purified isolates of Pediococcus acidilactici, Pediococcus pentosaceus, and Lactobacillus plantarum were grown-up in separate fermenters using standard anaerobic submerged liquid fermentation protocols. The individual organisms were recovered from the fermenters via centrifugation, mixed together in equal proportions on a weight basis, then added to the following mixture: 1 part inulin, 2.2 parts isolated soy protein, 8 parts rice flour with 0.25% w/w sodium chloride, 0.045% w/w Calcium carbonate, 0.025% w/w Magnesium sulphate, 0.025% w/w Sodium phosphate, 0.012% w/w Ferrous sulphate and 29.6% water. This mixture was allowed to ferment for up to 5 days at 30° C. Upon completion of the fermentation, the entire mixture was freeze dried to a moisture content less than 5%, ground to an average particle size of 295 microns, with 60% of the product in the size range between 175-840 microns, and homogenized. The final microbial concentration of the powdered product is between 10⁹ and 10¹¹ CFU/g.

Submerged Liquid Fermentation

Individual, purified isolates of Pediococcus acidilactici, Pediococcus pentosaceus and Lactobacillus plantarum were grown-up in separate fermenters using standard anaerobic submerged liquid fermentation protocols. After fermentation the individual cultures were filtered, centrifuged, freeze dried to a moisture level less than about 5%, then ground to a mean particle size of 295 microns, with 60% of the product in a size range between 175-840 microns. The individual dried microbial cultures were then mixed in equal proportion by weight to obtain the microbial composition of the present invention. The final microbial concentration of the mixed powdered product is between 10⁹ and 10¹¹ CFU/g.

### Example 2

### Formulation of Water Treatment Product

The following Water Treatment formulations were prepared by dry blending the ingredients in a ribbon blender (all percentages are by weight):

| | COMPOSITIONS | | | | | | | |
|---|---|---|---|---|---|---|---|---|
| Ingredients | A | B | C | D | E | F | G | H |
| Microbial | 5 | 5 | 10 | 10 | 25 | 25 | 50 | 50 |
| Composition | | | | | | | | |
| from Example 1 | | | | | | | | |
| Monohydrate | 95 | | 90 | | 75 | | 50 | |
| Dextrose | | | | | | | | |
| Nutri-Sure ™ | | 95 | | 90 | | 75 | | 50 |

### Example 3

### Formulation of Animal Feed Products—Coating

The dried microbial mixture of Example 2H is formulated into animal feed pellets (shrimp, poultry, swine, and cattle) via the following methods: 10 grams of low melting grease (e.g. hydrogenated soybean oil with m.p. of 47-48° C.) are heated to just slightly above the melting point (50° C.). Once all the grease is melted, 0.01 to 1 gram of the dried, powdered microbial composition from Example 2H is dispersed in the melt with rapid stirring. 95 grams of animal feed pellets are then quickly added to this melt and rapidly stirred to achieve homogeneous coating. The pellets are allowed to air dry overnight at room temperature. The final microbial activity of the coated pellet is between 10⁷ and 10⁹ CFU/g.

Alternatively, low melting grease (e.g. hydrogenated soybean oil with m.p. of 47-48° C.) is added to a tank and heated to 50° C. while stirring. The melted grease is sprayed onto a stirred bed of feed pellets heated with forced air to about 45° C. The final weight of grease ranges from 1 to 5% w/w. The dried microbial composition from Example 2H is added to the grease coated feed at weights between 0.01 and 1% w/w, the heated air flow is turned off, and the bed allowed to mix and cool until it reaches ambient temperature.

### Example 4

### Formulation of Animal Feed Products—Extrusion

The following feed formulations were prepared:

| Ingredient | COMPOSITIONS | | | | | |
|---|---|---|---|---|---|---|
| Composition (%) | A | B | C | D | E | F |
| Fishmeal | 26 | | 28 | 6 | 5 | 26 |
| Dehydrated Fish | | 20 | | | | |
| Solubles | | | | | | |
| Shrimp head meal | | 13 | | | | 5 |
| Shrimp shell meal | | | 10 | | | 5 |
| Wheat Flour | 27 | 21 | 10 | 16 | 8 | |
| Wheat gluten | | 15 | | | | |
| Soybean oil cake | 30 | 5 | 15 | 46 | 62 | 26 |
| Composition of | 5 | 20 | 10 | 10 | 15 | 5 |
| Example 1 | | | | | | |
| Rice Bran | | | 20 | | | |
| Canola Meal | | | | 10 | | |
| Tapioca powder | | | | | | 20 |
| Fish Oil | 2 | | | 4 | | 3 |
| Vitamin mix | 1 | 1 | 1 | 1 | 1 | 1 |
| Mineral mix | 0.5 | 0.5 | 0.5 | 0.5 | 0.5 | 0.5 |
| Binder | 0.5 | 0.5 | 0.5 | 0.5 | 0.5 | 0.5 |
| Water | 8 | 4 | 5 | 6 | 8 | 8 |

The ingredients for each formula were mixed together, heated to 120-150° C., then conveyed to a Wenger TX52 Twin Screw extruder with the screws setup in a conveying configuration (low shear, low friction). The paste that is created is pushed through a die having 3 mm openings. The extrudate is cut into 10 mm lengths using a four blade rotating knife. The resulting pellets are collected, cooled, and assayed for moisture. Typical moisture levels are below 10%. The microbial activity of the final composition is between 10⁵-10⁹ cfu/g.

### Example 5

### EMS Challenge Study

Shrimp feeding studies were conducted with the bacterial composition of Example 1:

Starting with PL10's, weight 0.005 grams in a zero water exchange system. Stocking density was 3384 shrimp/m2/tank.

Treatments

Control (no added biology)

0.25 mg/L of the composition from Example 1

25 mg/L of the composition from Example 1

The bacterial composition of Example 1 was added daily as a liquid (dried composition dissolved in water) during the morning feeding.

All tanks were fed 40% protein diets based on a diminishing FCR through days 0-5. During days 0-3, in addition to the feed called for in the FCR, 9 g of 45% protein standard reference diet (SRD) were added daily.

Feed protein rate and FCR were evaluated daily taking into consideration remaining feed in the tank, water quality, and biofloc level.

A zero water exchange raceway system was used.

After 53 days the study was terminated. Shrimp from all exposure groups (average weight 0.75 g) were harvested for testing in an EMS challenge study. EMS is caused by Vibrio parahaemolyticus (producing an infectious agent causing Acute Hepatopancreatic Necrosis Disease (APHND)). A total of 200 shrimp from the group exposed to the high dose of Example 1 composition were stocked into five 90 L tanks (40/tank). A total of 140 animals from the group exposed to the low dose of Example 1 composition were stocked into another set of four 90 L tanks (35/tank), and 120 animals from the control were stocked into three 90 L tanks (40/tank). Two tanks were stocked with 20 SPF (specific pathogen free) shrimp which had been reared as an additional control group.

All aquaria were outfitted with aeration and an oystershell filter, and were covered with a plastic sheet to reduce the risk of cross contamination. Additionally, the negative control tanks were kept isolated in a separate building and fed before the V. parahaemolyticus challenge tanks

The composition of Example 1 was provided in powdered form. The low dose group was exposed to 2.5 mg/L daily and the high dose group was exposed to 25 mg/L daily. The feed was a 23% protein shrimp pellet. All tanks were maintained on the appropriate dose of biology and feed while the animals recovered from shipping stress and for the duration of the challenge study.

The application of the composition from Example 1 was performed on the tanks each day while feeding. On the day that Vibrio parahaemolyticus was introduced into each tank (day 0 post-infection), the bacterial composition was added to each tank approximately 2 hours before the Vibrio challenge.

On day 0 of the Vibrio challenge, all challenge aquaria were fed a commercially pelleted diet (Rangen 40% protein) which had been soaked in a broth containing the V. parahaemolyticus at an optical density of 1.71 with 7×10⁸ colony forming units. On the afternoon of day 0, all Vibrio challenged aquaria were fed a second dose of the same V. parahaemolyticus broth, but this time the broth was added to the original feed diet.

All aquaria were checked once a day for moribund and dead animals. Moribund animals were preserved in fixative to confirm infection by histopathology and dead animals were frozen. The study was terminated after 10 days with live animals counted as survivors.

| Bacterial Composition | | |
|---|---|---|
| Dose | Tank Definition | % Survivability |
| No added Biology | Negative | 90.00% |
| Feed Only | Control | |
| | No AHPND | |
| | Challenge | |
| High Bacterial | Negative | 92.50% |
| Composition Dose | Control | |
| + | No AHPND | |
| Feed | Challenge | |
| Low Bacterial | AHPND | 37.10% |
| Composition Dose | challenge | |
| High Bacterial | AHPND | 49.40% |
| Composition Dose | challenge | |
| No added Biology | Positive Control | 5.00% |
| Feed and AHPND | AHPND | |
| | Challenge | |

Histological data on moribund shrimp confirmed infection by V. parahaemolyticus, and PCR analysis of the water in the negative controls confirmed presence of the pathogen. However, PCR analysis of the water in tanks with the composition from Example 1 was unable to detect the presence of V. parahaemolyticus—suggesting that the composition of the invention could also be inhibiting Vibrio growth.

DNA was extracted from samples of stock shrimp and shrimp from each treatment tank at the end of the growth experiment and taxonomic profiles of bacteria in the samples were created using a TRFLP protocol. Bacterial profiles were compared between treatments to look for significant differences.

The bacterial profile from stocking shrimp was significantly different from all the shrimp after growth in tanks Replicate samples showed a fair amount of variation in spite of 5 shrimp being analyzed per tank. When treatments were pooled into two categories, Shrimp fed the composition of Example 1 versus Shrimp without exposure, a significant difference was detected (p=0.026) between the two groups. This result shows that the bacterial composition of Example 1 significantly altered the microbial community present in shrimp gut.

### Example 6

### In Vitro Pathogenic Vibrio Inhibition Studies

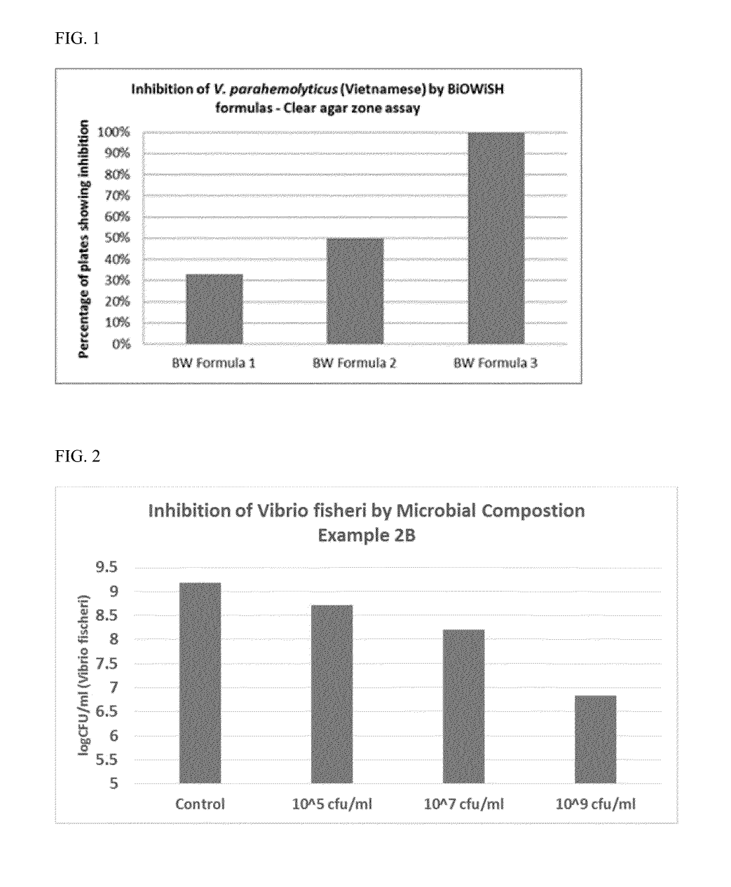

To further confirm that the compositions of Example 1 could be inhibiting Vibrio growth, and thereby, increasing shrimp resistance to EMS, the compositions were screened evaluated for their ability to inhibit the growth of different strains of the EMS species (V. parahaemolyticus) that had been isolated previously from global sourcing. The results demonstrate significant inhibition of Vibrio growth by the subject bacterial compositions (FIG. 1).

### Example 7

### Expanded Microbial Composition

A composition comprising the bacterial strains from Example 1 and additional microbes selected for their ability to provide additional antibacterial/antimicrobial benefits was designed using a fermentation process similar to that described in Example 1:

Bacillus and Paenibacillus Species

Individual starter cultures of Bacillus subtilis, Bacillus amyloliquefaciens, Bacillus licheniformis, Bacillus pumilus, Bacillus coagulans, Bacillus megaterium, and Paenibacillus polymyxa, were grown according to the following general protocol: 2 grams nutrient broth, 2 grams AmberFerm (yeast extract) and 4 grams Maltodextrin were added to a 250 ml Erlenmeyer flask. 100 milliliters distilled, deionized water was added and the flask stirred until all dry ingredients were dissolved. The flask was covered and placed for 30 min. in an Autoclave operating at 121° C. and 15 psi. After cooling, the flask was inoculated with 1 ml of one of the pure microbial strains. The flask was sealed and placed on an orbital shaker at 30° C. Cultures were allowed to grow for 3-5 days. This process was repeated for each of the microorganisms in the mixture. This process provided starter cultures of each organism which were then used to prepare larger scale fermentations.

Individual fermenters were run under aerobic conditions at pH 7 at the temperature optimum for each species:

| Microbe | Temperature Optimum |
|---|---|
| Bacillus subtilis | 35° C. |
| Bacillus | 30° C. |
| amyloliquefaciens | |
| Bacillus licheniformis | 37° C. |
| Bacillus coagulans | 37° C. |
| Bacillus megaterium | 30° C. |
| Bacillus pumilus | 32° C. |
| Paenibacillus polymyxa | 30° C. |

Each fermenter was run until cell density reached 10¹¹ CFU/ml, on average. The individual fermenters were then emptied, filtered, and centrifuged to obtain the bacterial cell mass which was subsequently dried under vacuum until moisture levels dropped below 5%. The individual microbes were then mixed in equal proportion to obtain a final, dried product with microbial count of 10⁹-10¹¹ CFU/g. This dried bacillus product was then mixed with the lactobacillus mix of Example 1 at equal weight to obtain the final microbial product which had an aerobic plate count of 10⁹-10¹¹ CFU/g.

### Example 8

### In Vitro Inhibition Assays for Vibrio Species

Preparation: (1) A 1.0 g sample of the microbial mixture of Example 2B was added to 100 mLs MRS broth and incubated for 48 hours at 35° C., 150 RPM. Successive aliquots of the resulting turbid media were aseptically added to vials of sterile MRS broth with sterile serological pipettes until the targeted CFU titer was achieved (use the standard OD600 value of 1.0 ABS=8.0×10⁸ CFU/mL). (2) A flask containing 90 mLs of sterile photobacterium broth (PBB) was inoculated with a loopful of Vibrio fischeri taken from a plate of photobacterium agar (PBA). The flask was cultured for 24 hours at 25° C., 150 RPM. (3) 12 flasks of 80 mL PBB and four plates of PBA per planned replicate were prepared according to manufacturer's instructions. The PBA plates were allowed to cure for 48 hours before use. And (4) Six vials of sterile, lx Phosphate Buffered Saline (PBS) per planned replicate were prepared.

Assay setup: A 10 mL aliquot of the Vibrio culture and 10 mLs of the microbial treatment (of targeted titer) were aseptically added to 80 mLs sterile PBB. The flask was capped and shaken at 25° C., 150 RPM for 24 hours.

Serial dilutions: (1) a 1 mL aliquot was aseptically removed from each culture flask and added to the first 9 mL vial of PBS. The vial was vortexed on high for 5 seconds to ensure adequate mixing. 1 mL was removed from this vial using a sterile pipette and the procedure repeated until six dilution vials were completed. And (2) A 100 μL aliquot was taken from each of the dilution vials and plated on separate plates of cured PBA. The samples were spread on the agar with a flame-sterilized spreader. The plates were allowed to sit upright for 15 minutes to absorb the inoculate, then inverted and incubated at 25° C. for 48 hours before counting colonies.

Results are shown in FIG. 2.

We claim:

1. An antibacterial composition comprising a mixture of bacteria selected from the family Lactobacillaceae.

2. The composition of claim 1, wherein the mixture of bacteria comprises a species selected from the genus Pediococcus and Lactobacillus.

3. The composition of claim 2, wherein the mixture of bacteria comprises equal amounts of Pediococcus acidilactici, Pediococcus pentosaceus, and Lactobacillus plantarum and each of the bacteria in the mixture is individually anaerobically fermented, harvested, dried, and ground to produce a powder having a mean particle size of about 200 microns, with greater than 60% of the mixture in the size range between 100-800 microns.

4. The composition according to claim 3, wherein the mixture of bacteria further comprises a species selected from the genus Bacillus.

5. The composition of claim 4, wherein the Bacillus species is Bacillus subtilis 34KLB.

6. The composition of claim 5, wherein the composition comprises about 2% by weight mixture of bacteria comprising equal amounts of Pediococcus acidilactici, Pediococcus pentosaceus, and Lactobacillus plantarum, and about 0.15% of Bacillus subtilis 34KLB by weight.

7. The composition of claim 6, wherein each bacteria in the mixture is individually fermented under conditions optimum for growth, harvested, dried, and ground to produce a powder having a mean particle size of about 200 microns, with greater than 60% of the mixture in the size range between 100-800 microns.

8. The composition of claim 6, further comprising about 4% diatomaceous earth.

9. The composition of claim 6, further comprising at least 95% rice bran or Nutri-Sure or combination thereof.

10. The composition of claim 8, further comprising at least 90% dextrose monohydrate.

11. The composition of any one of the preceding claims, wherein the composition has a moisture content less than about 5%; and a final bacterial concentration of about between 10⁵-10¹¹ colony forming units (CFU) per gram of the composition.

12. The composition of claim 6, further comprising an inert carrier.

13. The composition of claim 12, wherein the inert carrier is dextrose monohydrate, anhydrous dextrose, dendritic salt, rice bran, soybean meal, wheat bran, rice hulls, oat bran, Nutri-sure or a mixture thereof.

14. The composition of claim 12, wherein the inert carrier is at a concentration of about between 75-99% (w/w).

15. The composition of claim 6, further comprising a drying agent.

16. The composition of claim 15, wherein the drying agent is diatomaceous earth.

17. The composition of claim 15, wherein the drying agent is at a concentration of about 1-10% (w/w).

18. The composition of claim 17, wherein the drying agent is at a concentration of about 4% (w/w).

19. A method of treating a body of water comprising contacting said body of water with the composition according to the composition of claim 1.

20. The method of claim 19, wherein the composition is dosed between 0.001 and 100 mg/L.

21. The method of claim 19, wherein the body of water is a lagoon, pond, lake, or raceway system used to raise an aquatic organism.

22. The method of claim 21, wherein the aquatic organism is shrimp or fin fish.

23. The method of claim 22, wherein the fin fish is catfish, tilapia, salmon, carp, sea bass, or cod.

24. The method of according to claim 19, wherein treating the body of water results in a decrease in pathogenic bacteria.

25. The method of claim 24, wherein the pathogenic bacteria is Vibrio, Escherichia, Listeria, or Salmonella.

26. The method of claim 25, wherein the pathogenic bacteria is V. cholera, V. parahaemolyticus, V. harveyi, V. vulnificans, or V. fischeri.

27. The method of claim 24, wherein treating the body of water results in at least a 2-log reduction in the population of pathogenic bacteria.

28. A method for treating an aquaculture system infected with V. parahaemolyticus, wherein the system is dosed with between 0.001 and 100 mg/L of the composition of claim 1.

29. The method of claim 28, wherein treating the aquaculture system includes a minimum 2-log reduction in the population of V. parahaemolyticus.

30. A method for managing Early Mortality Syndrome in shrimp farming, wherein the shrimp are exposed to the composition of claim 1 at a concentration of between 0.001 and 100 mg/L.

31. The method of claim 30, wherein the shrimp is exposed to the composition by dosing the composition directly to the water.

32. The method of claim 30, wherein the shrimp is exposed to the composition through a feed particle admixed with or coated by the composition.

33. The method of claim 32, wherein the feed particle is produced through a standard pelleting process.
